(19) United States
(12) Patent Application Publication
(10) Pub. No.: US 2021/0193284 A1
(43) Pub. Date: Jun. 24, 2021

(54) A METHOD AND A SYSTEM FOR MONITORING AND CONTROLLING THE USE OF DRUGS AND/OR MEDICAL SUPPLIES IN A PHARMACEUTICAL DISTRIBUTION PROCESS

(71) Applicant: NEWICON OY, Kuopio (FI)

(72) Inventors: Ossi PARVIAINEN, Kuopio (FI); Juha LAAKSONEN, Kuopio (FI)

(21) Appl. No.: 17/058,400

(22) PCT Filed: May 28, 2018
(86) PCT No.: PCT/FI2018/050403

§ 371 Date: Nov. 24, 2020

(51) Int. Cl. G16H 20/13; G16H 40/67; A61G 12/00

(52) U.S. Cl. CPC G16H 20/13; A61G 2205/60; A61G 12/001; G16H 40/67

(57) ABSTRACT

The invention relates to a method wherein drug packages stored in drug storage devices, or drugs transferred from drug packages to various storage containers for storage, are arranged in transfer containers equipped with remotely readable tags, and in which method and system, location data on the transfer containers equipped with remotely readable tags are recorded, upon situations of changing the location, in one or more data processing devices controlling the storage and dispensing data on the drugs and/or medical supplies, by readers for the remotely readable tags, connected to the data processing device/devices. Consequently, e.g. data on the locations, and on the quantities in different locations, of the drugs described to patients, and/or the medical supplies used in treatments of the patients, are available to the storage device and to other devices connected to the system. The invention relates also to a system according to the method of the invention.

### FIELD OF THE INVENTION

The invention relates to a method and a system for monitoring and controlling the use of drugs and/or medical supplies in a pharmaceutical distribution process.

### BACKGROUND OF THE INVENTION

The operation of hospitals, health clinics and private health centres requires continuous close monitoring and control of drugs and supplies used in medication and other treatments, in the different steps of their storage, dispensing and/or use. Therefore, these functions and related processes also play a key role in securing patient safety as well as in controlling losses of drugs and medical supplies, and the costs involved. Storing, transferring, and monitoring the use of drugs and medical supplies still involve a number of manual work stages, wherein e.g. ensuring that correct drugs are transferred from drug stor-ages and cabinets further is based on the vigilance and diligence of the person performing each work stage.

In hospitals and health clinics, drugs to be administered to patients are nor-malty dispensed once per 1 to 7 days, according to the prescription and/or the instructions on the drug package. Before the dispensing, the drug packages are stored and recorded in a storage, from which they are transferred, either in the original packages or packed in separate containers, such as bags or jars. These may be transferred to the storage and transfer systems, such as drug cabinets or trolleys, of the wards. At the stage of dispensing the drugs, a nurse has to separately make sure that they comply with the dose according to the prescription, and that the drugs are in an appropriate condition. Moreover, the nurse often prepares e.g. ampoules, drugs to be administered as an injection or intravenously, to be ready for administration. After that, the nurse goes to the patients and administers the drugs. Logging systems also make it possible for the nurse to dispense drugs in an order deviating from the normal dispensing process.

### BRIEF SUMMARY OF THE INVENTION

It is an aim of the invention to provide a method in which the monitoring and the control of drugs and medical supplies in the different steps of their dispensing processes can be made more reliable and flexible than before, and the probability of errors can be reduced further to a level lower than before. Moreover, it is an aim of the invention to present a system for applying the method according to the invention.

The aim of the invention is achieved by a method and a system, in which drug packages stored in drug storage devices, or drugs transferred from drug packages to various storage containers for storage, are arranged in transfer containers equipped with remotely readable tags, and in which method and system, location data on the transfer containers equipped with remotely readable tags are recorded, upon situations of changing the location, in one or more data processing devices controlling the storage and dispensing data on the drugs and/or medical supplies, by readers for the remotely readable tags, connected to the data processing device/devices. Consequently, e.g. data on the locations, and on the quantities in different locations, of the drugs described to patients, and/or the medical supplies used in treatments of the patients, are available to the storage device itself as well as to other devices connected to the system according to the method of the invention. To put it more precisely, the method according to the invention is characterized in what will be presented in the independent claim 1, and the system is characterized in what will be presented in the independent claim 7. Dependent claims 2 to 6 present some preferred embodiments of the method according to the invention, and dependent claims 8 to 17 present some preferred embodiments of the system according to the invention.

The method and the system according to the invention have the advantage that an outdated drug product or dose which is possibly defective can be withdrawn before it is administered to a patient. Also, a drug which has not been administered because of a changed prescription or for another reason can be allocated to another use. Thus, it does not have to be disposed of, or for example encoded for a new storage location; the same encoding may also be applied to include data relating to access rights, origin, and new dispensing. This reduces the checking work which is time consuming and susceptible to errors. The remotely readable encoding relating to the transfer containers also increases medical safety. The encoding and the logging-in by the user make it possible to see who has moved an encoded container to make its contents accessible.

### DESCRIPTION OF THE DRAWINGS

In the following, the invention will be described in more detail with reference to the appended drawings, in which

FIG. 1 shows a drug cabinet belonging to a system according to the method of the invention, in a slanted view from above;

FIG. 2 shows a transfer container for use in a system according to the method of the invention, in a slanted view from the side;

FIG. 3 shows another transfer container for use in a system according to the method of the invention, in a slanted view from the front; and

FIG. 4 shows a dispensing trolley belonging to a system according to the method of the invention.

### DETAILED DESCRIPTION OF PREFERRED EMBODIMENTS OF THE INVENTION

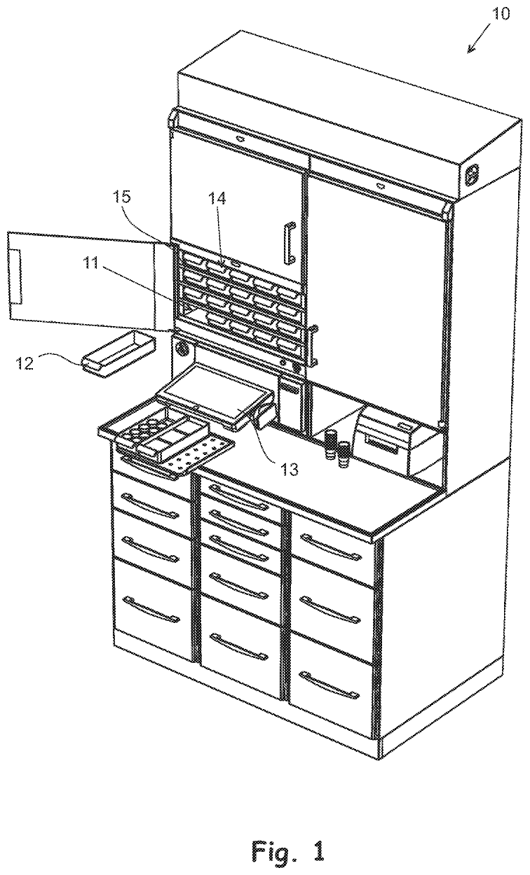

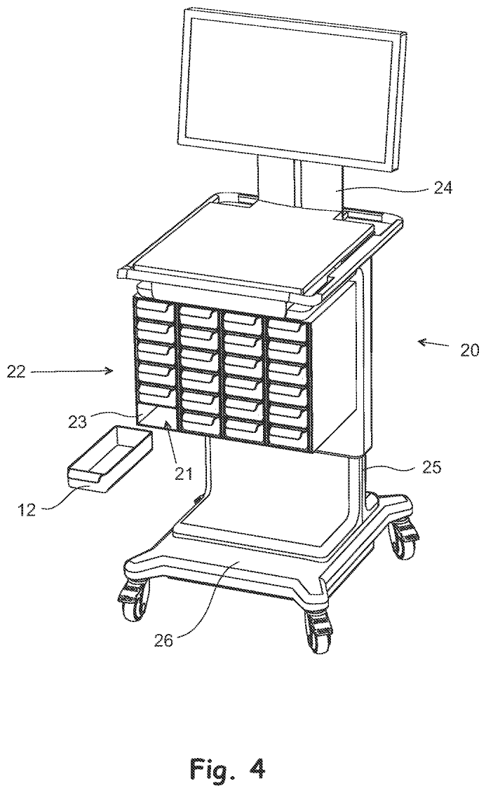

In the simplest way, the method according to the invention is implemented by a system comprising at least one storage device for storing drugs and/or medical supplies. According to the method of the invention, the drugs and/or medical supplies are stored in transfer containers placed in storage locations in the storage device. The storage device may be, for example, a drug cabinet 10 as shown in FIG. 1, a dispensing trolley 20 as shown in FIG. 4, a medical bag, a waist bag, or another device. The transfer containers 12 used in the method may be placed in any storage location reserved for such transfer containers in the storage device. Furthermore, the storage device according to the method and the system of the invention comprises at least one reader for remotely readable tags, as well as a data processing device or a communication device via which the storage device communicates with a data processing device belonging the system according to the method of the invention. The data processing device may be, for example, a portable computer, a tablet computer, or another data processing device suitable for the purpose, such as a program-mable mobile data processing device connected to the storage device in a wired or wireless manner, for controlling its functions. The reader for remotely readable tags is a device capable of reading one type or some different types of remotely readable tags, such as RFID and/or NFC tags. The transfer containers 12 are boxes or cases enclosed by at least one wall and having an inner space in which the drugs and/or medical supplies to be placed in the transfer container 12 can be inserted via at least one wall or a part of a wall of the transfer container 12 that is open or openable and closable by a lid. Furthermore, the transfer containers 12 are provided with a remotely readable tag, such as an RFID or NFC tag containing a unique ID code for identifying each transfer container 12 and for distinguishing it from other transfer containers. The remotely readable tag is provided on at least one wall or openable and closable lid of the transfer container. The function of the remotely readable tag is to enable the identification of the transfer container by devices included in storage devices for reading remotely readable tags, in order to monitor drugs and medical supplies and to control the dispensing process as desired.

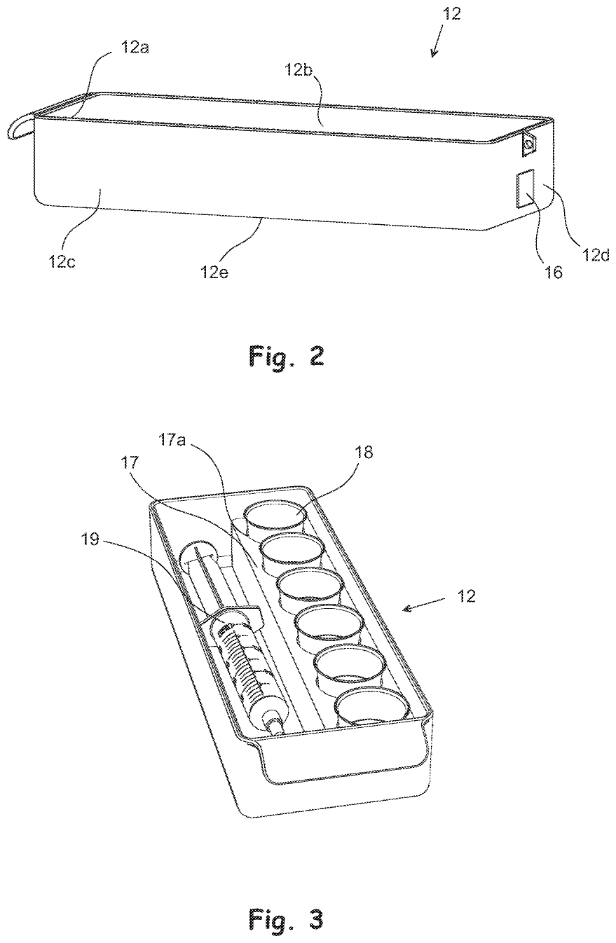

FIG. 1 shows a drug cabinet 10 which is a storage device according to a method of the invention. It contains several storage locations 11 for storing transfer containers 12 shown in FIG. 2 or 3, and a data processing device 13 including a memory for storing data on the drugs and/or medical supplies to be stored in the storage containers 12, and the ID codes of the transfer containers, as well as for a computer program controlling the handling and dispensing of drugs. Furthermore, the drug cabinet shown in FIG. 1 comprises a reader for remotely readable tags, by which the remotely readable tags 16 of the transfer containers 12 can be read, for identifying and distinguishing them from other transfer containers 12. The reader for remotely readable tags is connected to the data processing device 13 so that the ID code of the remotely readable tags 16 can be read into the memory of the data processing device and used for controlling the dispensing process by a computer program installed in the data processing device 13 or in another data processing device connected to it and belonging to the system.

The storage locations 11 in the drug cabinet 10 are arranged in a shelving unit 14 closable by a door, several storage locations 11 being provided next to each other on each shelf 15 of the shelving unit 14. In this embodiment, the width of the shelves 15 is designed so that each shelf 15 accommodates a given number of transfer containers 11 placed tightly next to each other. The distance between the shelves 15 in the height direction of the drug cabinet 10 is approximately equal to the height of the transfer containers 12, so that drugs or medical supplies placed in a transfer container 12 next to an empty storage location 11 cannot be accessed via the empty storage location 11 next to the transfer container 12 without removing the transfer container 12 from its storage location 11.

FIG. 2 shows a transfer container 12 for use in a drug cabinet 10 according to FIG. 1. In this case, the transfer container 12 is a box made of e.g. plastic, thin sheet metal or another suitable material, open at the top, and having a front end wall 12a, side walls 12b and 12c, a rear end wall 12d, and a bottom 12e. The transfer container 12 comprises a remotely readable tag 16 which in this case is an NFC tag. It comprises an ID code which is readable by e.g. the reader for remotely readable tags, belonging to the drug cabinet 10. In this embodiment of the transfer container, the NFC tag 16 is placed on the rear end wall 12d, as shown in FIG. 2. Thus, a reading head of the reader, provided for each storage location and placed between the shelves 15 of the shelving unit 14 on the rear wall of the drug cabinet, can read the remotely readable tag 16 on the transfer container 12 at any time when the transfer container 12 is inserted in a storage location 11 suitable for it in the drug cabinet 10.

FIG. 3 shows the inner space of the transfer container 12, formed by the front end wall 12a, the side walls 12b and 12c, the rear end wall 12d, and the bottom 12e. In this case, the bottom 12e of the transfer container is provided with a set of compartments 17 on its first side, extending from the bottom upwards on the side of the inner space of the transfer container 12, and comprising several recesses 17a for dispensing units at regular intervals. A number (for example six as shown in the figure) of dispensing units (drug cups) 18 can be placed in these recesses. The other side of the transfer container is reserved for a drug syringe 19 suitable for administering injectable drugs.

In the drug cabinet 10, the reader for remotely readable tags may comprise one reading head (not shown in the figures) which is manually movable with respect to the drug cabinet, for reading remotely readable tags on transfer containers. Furthermore, in this case, each storage location 11 of the drug cabinet 10 is equipped with a reading head placed on the rear wall of the drug cabinet and configured to read the remotely readable tag of the transfer container 12 (e.g. at given intervals) when the transfer container 12 is inserted in any of the storage locations 11 of the drug cabinet. Thanks to this arrangement, the data processing device 13 of the drug cabinet 10 receives constant real-time information on which transfer container 12 is inserted in any particular storage location 11 of the drug cabinet. Thanks to this, it is insignificant into which storage location a given transfer container 12 is inserted, because the computer program can constantly update the location data on the transfer containers 12 and thereby, for example in a dispensing situation, guide the user always to pick up the desired drugs and/or medical supplies from the correct transfer container 12. Moreover, drugs and/or medical supplies stored in a transfer container 12 inserted in a given storage location 11 cannot be removed without leaving a log entry in the memory of the data processing device 13 of the drug cabinet 10, because each time a transfer container 12 is drawn out to a given extent from its storage location 11, or removed from the storage location, the data processing device 13 will record the absence of the remotely readable tag 16 of the transfer container 12 from the vicinity of the reading head, i.e. record the change relating to the transfer device 12. In such a case, the computer program may, for example, include the time and the data on the user who was logged-in on the computer program of the drug cabinet (e.g. for opening the locking of the doors of the drug cabinet) and moved the transfer container 12, in the combined data recorded with the ID code corresponding to the transfer container.

The manually movable reading head of the drug cabinet 10 may include a bar code and/or a QR code reader in addition to the RFID and NFC reader. The reading head for NFC tags may be used for identifying the transfer container 12 when drugs and/or medical supplies are being added to or removed from a transfer container 12. After that, bar and/or QR codes on drug packages can be read by the bar and/or QR code reader of the reading head. By reading the bar and/or QR codes, the data processing device 13 receives the data on the drugs to be added to a given transfer container 12, their quantity, and other data (e.g. expiry date). The bar or QR code may be configured to control the data processing device 13 to retrieve data on the drugs to be added to (or removed from) the transfer container directly from a database maintained by the respective pharmaceutical manufacturer and simultaneously, for example, to authenticate the drug on the basis of its serial/production number.

In this case, the data processing device 13 is a tablet computer with a touch sensitive screen. A computer program is installed in the data processing device 13 to control the filling of transfer containers 12 in the drug cabinet 10 with drugs and/or medical supplies. The computer program also controls the arrangement of the transfer containers 12 in the storage locations 11 in the drug cabinet. For this, the storage locations 11 may be equipped with indicating means (e.g. LEDs) by which the data processing device 13 may indicate to the user which storage location 11 a transfer container should be placed in, and particularly which storage location 11 a specific transfer container 12 (containing drugs and/or medical supplies ordered by the user) can be found in, to be transferred to another storage device or to be removed from the drug cabinet 10, for removing some or all of the drugs and/or medical supplies from the transfer container 12.

FIG. 4 shows another storage device 20 for use in a method according to the invention. The storage device 20 shown in FIG. 4 is a dispensing trolley. The dispensing trolley 20 shown in FIG. 4 comprises several storage locations 21 for transfer containers 12 shown in FIGS. 2 and 3. In the dispensing trolley 20 shown in FIG. 4, the storage locations 21 are provided in an organizer 22 with several compartments 23 designed according to the outer dimensions of the transfer containers 12 and arranged on top of each other and side by side. The organizer 22 is dimensioned so that a transfer container 12 can be inserted in a compartment 23 of the organizer 22 via openings on the front side of the organizer 22. Thus, new transfer containers can be added or, for example, transfer containers 12 stored in the drug cabinet 10 can be moved to the dispensing trolley 20 shown in FIG. 4 when drugs are to be dispensed to patients, and/or when medical supplies stored in the transfer containers 12 are to be transferred to another location farther away (e.g. to another ward in a hospital). The organizer 22 may be equipped with intermediate walls between the different compartments 23, to prevent access, via empty compartments on top or by the side, to a storage location 21 which already accommodates a transfer container 12.

The dispensing trolley 20 of FIG. 4 further comprises a data processing device 24 and a reader for remotely readable tags, for reading the remotely readable tags of the transfer containers 12. in this case, the data processing device 24 is a computer integrated in the dispensing trolley 20 and equipped with a memory for data on the transfer containers 12 and the drugs and/or medical supplies to be stored in them, as well as for a computer program controlling the process of dispensing the drugs and/or medical supplies. Furthermore, the data processing device 24 may comprise a display, a keyboard and a mouse for applying and controlling, for example, the computer program installed in the data processing device for controlling the process of dispensing the drugs and medical supplies. The reader for remotely readable tags reads the remotely readable tags 16 on the transfer containers 12 and identifies the transfer container 12 transferred into or from the dispensing trolley 20. The computer program installed in the data processing device 24 controls the handling of transfer containers 12 in the dispensing trolley 20. The data processing device 24 may also be connected to the data processing device 13 of the drug cabinet 10, for example via a wireless local area network (WIFI) or another suitable communication, and thus utilize the data and/or the computer program in the data processing device 13 of the drug cabinet 10. The computer program of the dispensing trolley 20 may also update the data in the computer program in the data processing device 13 of the drug cabinet 10 so that, for example, the location data of the transfer containers 12 possibly transferred from the drug cabinet 10 to the dispensing trolley 20, as well as the stock balance of the drugs and/or medical supplies, are automatically updated in the data processing device 13 of the drug cabinet 10 as well. Instead of the data processing device, the dispensing trolley 20 may only be equipped with a communication device (a wireless router, or the like), by which the dispensing trolley communicates, constantly or intermittently in a wireless manner (e.g. via a wireless local area network or the like), with a data processing device in the system (such as a data processing device connected to an automated storage system, a drug cabinet or another dispensing trolley, comprising a computer program operating according to the method for controlling the process of dispensing drugs and/or medical supplies).

Also, the reader of the dispensing trolley 20 for remotely readable tags may also comprise a reading head for reading remotely readable tags other than the remotely readable tags on the transfer containers. For example, when drugs and/or medical supplies in the transfer containers 12 are being carried by the dispensing trolley 20 to patients, it can be used for reading a remotely readable patient tag for identifying the patient. The reader for remotely readable tags may comprise one manually movable reading head connected to a data processing device (in a wired or wireless manner). In addition to the movable reading head, a reading head is provided on the rear wall of each compartment in the organizer for reading the remotely readable tag 16 on the rear wall of the transfer container when the transfer container is inserted in the storage location 21. Thanks to this feature, when transfer containers 12 are being added to the storage locations 21 in the dispensing trolley 20, it is insignificant which storage location 21 each transfer container 12 is placed in, because the reading head for remotely readable tags in the storage location 21 always reads the remotely readable tag 16 on the transfer container 12, whereby the data processing device 24 is always informed of the storage location 21 in which a given transfer container 12 is placed. This makes the processes of dispensing drugs and/or medical supplies simpler and faster.

Also, the storage locations 21 of the dispensing trolley 20 may be equipped with indicating means for indicating the storage location 21 in which a given transfer container 12 is placed in the dispensing trolley 20. For example, when the drugs placed in the transfer containers 12 of the dispensing trolley 20 are being dispensed in inpatient wards of a hospital or a health clinic, the indicating means may be configured to indicate the transfer container 12 containing the drug to be administered next to a given patient after the patient's data have been entered in the data processing device (e.g. by reading a remotely readable tag on the patient and/or the patient's bed by the reader for remotely readable tags in the dispensing trolley 20). The indicating means are, for example, LEDs turned on and off by the data processing device 24. Furthermore, if these LEDs are polychromatic (have changing colours), the location of the transfer container 12 may be indicated, for example, by changing the colour of the LED to indicate the storage location 21 where the desired transfer container 12 can be found. Coloured LEDs may also be applied to indicate other information relating to the transfer containers 12, for example shortage or expiry of the drugs and/or medical supplies in the transfer container 12.

The dispensing trolley 20 shown in FIG. 4 naturally has a frame 25 made of e.g. a metal, comprising a lower trolley 26 movable on wheels, on top of which the organizer 22 comprising the storage locations 21 is placed on a height level suitable for the user so that it is as easy and ergonomic as possible to move the dispensing trolley 20 and to handle the transfer containers 12.

In the drug dispensing process according to the method of the invention, transfer containers and storage devices of the above described types are utilized. For example, in an embodiment of the method according to the invention, drugs in drug packages and/or various medical supplies (such as drug syringes, drug vials, drug cups, surgical instruments, dressings, etc.) are placed in transfer containers 12 shown in FIG. 2 or 3, to be stored in a drug cabinet 10 shown in FIG. 1. At this stage, the data on the drugs and/or medical supplies are entered in a computer program in a data processing device 13, which combines them with the ID code of the remotely readable tag 16 of the transfer container 12 so that the data can be printed out (e.g. on a display or on paper) by means of the ID code at any stage of handling the transfer container 12 by reading the ID code from the remotely readable tag 16 of the transfer container 12. The drug dispensing process according to the method, utilizing the drug cabinet 10 shown in FIG. 1, may continue for example as fol-lows: A drug package is ordered, for example, from an automated storage system containing drug packages. Its bar code is read, and it is placed in a transfer container 12 equipped with an ID code. The ID code of the transfer container 12 is read, and the transfer container 12 is placed in a vacant storage location 11 in the drug cabinet 10. Thus, the data processing device 13 of the drug cabinet 10 combines the ID code of the transfer container 12 and the code of the drug package. The dispensing trolley 20 of FIG. 4, in which exactly this drug is needed to be distributed, is brought next to the drug cabinet 10. The transfer container 12 is picked up from the storage location 11 of the drug cabinet 10 and transferred to a vacant storage location 21 in the dispensing trolley 20.

Thus, the reader reads the ID code from the remotely readable tag 16 of the transfer container 12, whereby the data processing device 24 of the dispensing trolley 20 automatically updates the changed data on the transfer container 12 in the data processing devices of the drug cabinet 10 and the dispensing trolley 20, as well as all the other data processing devices connected to or communicating with the system. After this, the nurse makes the drug dispensing rounds. Having arrived at the inpatient ward, the nurse selects a patient via a user interface, or applies the reader of the dispensing trolley 20 for remotely readable tags to read the patient's tag, or identifies the patient in another way as used in each case (e.g. on the basis of the room and bed number, or a name sign on the bed, by entering this data in the data processing device of the dispensing trolley). After entering the patient's data, the indication means of the dispensing trolley 20 indicate the storage location 21 in the dispensing trolley where the transfer container(s) 12 containing said patient's drugs are found. Thus, the data processing device and the computer program therein also register the drugs being administered by the nurse to the patient. When administering drugs contained in the transfer containers 12 to the patients, the nurse only needs to enter the quantity of the drugs administered. Thus, the data is automatically updated in the data processing device 24, which also updates the data, via a network connection, in the data processing device 13 of the drug cabinet 10 and possibly the drug stock balance of the hospital or health clinic (depending on the data systems). In other words, the complete drug distribution process in said hospital or health clinic can be monitored and controlled in real time, from the loading platform down to the administration to the patient. For the next patients, the dispensing of drugs continues in a way corresponding to that described above. After the dispensing rounds, the nurse brings the dispensing trolley back to the vicinity of the drug cabinet 10 and returns some or all of the transfer containers from the drug dispensing trolley back to the drug cabinet, for example depending on the drugs needed on the next drug dispensing round, and on the time of the next drug dispensing round.

The method and the system according to the invention may be implemented in a way deviating, in many respects, from the example embodiment presented above. For example, the system according to the invention may also comprise storage devices of types other than the above mentioned drug cabinet 10 or dispensing trolley 20. In an embodiment of the system according to the invention, the storage device is a “handling tray” separate from the drug cabinet or dispensing trolley, provided with transfer container seats in which the transfer containers may be placed for replenishment or emptying of dispensing units (e.g. dispensing cups). The transfer container seats of the handling tray may be equipped with indicating means controlling the replenishment of the dispensing units (e.g. placed in dispensing cup recesses within the transfer containers shown in FIG. 3) in the transfer containers with drugs to be removed from drug packages. When using such an arrangement, for example, the dispensing trolley may also be equipped with a corresponding handling tray, in which the transfer containers placed in the storage locations of the dispensing trolley are placed when drugs are dispensed to patients. At the stage of dispensing drugs to patients, the indicating means at the transfer container locations on the handling tray guide the nurse to pick up the dispensing unit containing the drugs intended for a given patient, for example in the case of a transfer container 12 of FIG. 3, from a given dispensing unit recess 19 in the transfer container. If the handling tray is separate from the other storage devices, it may comprise a separate data processing device and a reader for remotely readable tags.

In the process of dispensing drugs and/or medical supplies according to the invention, in which handling trays are utilized, the process of dispensing e.g. drugs according to the method of the invention may be the following: A nurse is at a drug cabinet 10 and applies the user interface of the computer program of the data processing device to select the dispensing of drugs for the morning round of the ward in question (e.g. cardiology ward). Thus, the computer program of the drug cabinet instructs the nurse to place an empty transfer container 12 (e.g. of the type shown in FIG. 3) in a transfer container seat on the handling tray. After this, the indicating means of the handling tray guide the nurse to place empty dispensing units (e.g. drug cups) in the dispensing unit recesses 19 (e.g. of the type shown in FIG. 3) in the transfer container. Next, the computer program controls the indication means to indicate which other transfer containers 12 should be taken out. The nurse picks up a transfer container containing a drug and places it in another transfer container location on the handling tray. After this, the indicating means guide the nurse to place a prescribed quantity of drugs in the dispensing units in the transfer container being handled. This step is repeated until all the drugs of the drug dispensing round have been dispensed in all the dispensing units in the different transfer containers. Next, the drugs are dispensed to patients. First, the transfer containers are transferred to a dispensing trolley (which may be e.g. a dispensing trolley 20 according to FIG. 4), after which the dispensing trolley 20 is moved to the ward where the dispensing is to be performed (this step might also be controlled by the data processing device of the dispensing trolley 20 by dis-playing the location of the ward on a map). After arriving at the first patient, the data of said patient are entered in the data processing device 24 of the dispensing trolley 20 in the above described way. At this stage, indicating means of the dispensing trolley 20 indicate the storage location of the transfer container 12 containing the drugs to be administered to the patient. After this, the transfer container 12 indicated by the indicating means of the storage locations 21 is removed from the storage location 11 in the dispensing trolley 20, and is placed on the handling tray of the dispensing trolley 20 (or on a separate handling tray which may be, for example, ready by the patient's bed). Next, the handling tray indicates which dispensing unit in the transfer container 12 contains the drugs to be administered to this patient. After the administration of the drugs, the transfer container 12 is returned to any of the vacant storage locations in the dispensing trolley 20 (and not necessary to the same storage location from which it was taken), after which the dispensing trolley 20 will automatically update the location data of the transfer container 12 by reading the remotely readable tag of the transfer container 12. When selecting the dispensing unit to be administered to the patient, the handling tray also updates the data on which drugs, and how much, have been administered to said patient. Thus it also updates the quantity of said drugs in the computer program of the dispensing trolley 20 and thereby also in the data processing device of the drug cabinet as well as in the drug stock database of the entire hospital or health clinic. If there are also other drugs to be administered to this patient, the dispensing trolley 20 will guide the nurse to dispense them in a similar way. The administration of drugs to other patients on the drug dispensing round in this ward (or in the next wards) is performed in a similar way as well. After that, the dispensing trolley 20 may be moved back to the vicinity of the drug cabinet 10 and, if desired, the transfer containers 12 may be returned to the drug cabinet 10, whereby the reader for remotely readable tags in the drug cabinet 10 will identify the transfer containers 12 and update their location data so that said transfer containers 12 are known to have been returned to the storage locations 11 in the drug cabinet 10. Again, in the step of returning the transfer containers 12, it is insignificant to which of the vacant storage locations in the drug cabinet 10 they are returned, because the reading heads of the reader for remotely readable tags in the storage locations 11 of the drug cabinet 10 identify the transfer containers 12 by their remotely readable tags when the transfer containers 12 are being inserted in the vacant storage locations 11. Furthermore, if the transfer containers 12 contain drugs which should be removed at the stage of returning, the drugs may be removed by placing such transfer containers 12 on a handling tray to indicate which dispensing units, single drugs or drug packages contained by the transfer containers 12 are to be removed and transferred elsewhere or to be, for example, disposed of in an appropriate way.

Also, the transfer containers may be implemented, in many respects, in ways different from the above described boxes open at the top. In some cases, the transfer containers may be boxes with an openable and closable lid. The shape of the transfer containers might also be e.g. cylindrical, polygonal, conical, adapted to the shape of the product stored in them, or even spherical, whereby their single wall is provided with an opening or an open section via which the drugs and/or medical supplies can be inserted in the transfer container. The transfer containers may also be equipped with internal intermediate walls or e.g. recesses formed on the bottom, for dispensing units, medical supplies, ampoules, etc. to be fitted in them.

The method and the system according to the invention are not limited to the above described example embodiments but may vary within the scope of the appended claims.

1. A method for monitoring and controlling the use of drugs and/or medical supplies in a pharmaceutical dispensing process, in which method drugs and/or medical supplies are transferred from a delivery package to at least one storage device (10, 20), from one storage device (10) to another storage device (20), and/or further to be dispensed to patients and/or to be used in the treatment of patients, and in which method:
the drugs and/or medical supplies to be moved to the storage device (10, 20) are placed in transfer containers (12) equipped with a remotely readable tag (16) and to be stored in the storage device (10, 20), the transfer containers (12) being containers which are separate from the storage device (10, 20) and from the product package of the drugs and/or medical supplies, and are removable from the storage device (10, 20);
the quantity and data of the drugs and/or medical supplies, and the ID code included in the remotely readable tag (16) of the transfer container are stored in the memory of the data processing device (13, 24) equipped with a computer program for controlling the process of dispensing the drugs and/or medical supplies;
the computer program combines the data of the drugs and/or medical supplies with the ID code of the remotely readable tag (16) of the transfer container (12) in such a way that the location and/or data of the drugs and/or medical supplies in the transfer container (12) can be checked and/or printed out at any stage of the dispensing process by reading the remotely readable tag (16) of the transfer container (12) by an ID code reader connected to the data processing device (13, 24) equipped with the computer program controlling the dispensing process, or another data processing device connected to the data processing device (13, 24) equipped with the computer program controlling the dispensing process;
when drugs and/or medical supplies are removed from or added into the transfer containers (12), the data and quantity of the drugs and/or medical supplies subject to the change are entered in the memory of the data processing device (13, 24) containing the computer program, by means of the computer program controlling the dispensing process.

2. The method according to claim 1, wherein the data on the drug are retrieved from a database containing the data on the drugs into the memory of the data processing device (13) equipped with the computer program controlling the dispensing process, by means of a bar code or a QR code on the drug package.

3. The method according to claim 1 or 2, wherein at least one storage device (10, 20), into which at least one transfer container is transferred, is a dispensing trolley (20) comprising at least one storage location (21), in which said at least one transfer container (12) to be placed in the dispensing trolley (20) can be placed; at least one separate reader for reading the remotely readable tag of at least one transfer container (12); and a data processing device (24) and a computer program installed in it, and/or a communicating device via which the data processing device (24) of the drug dispensing trolley communicates with the data processing device (13) controlling the dispensing process, for storing data on drugs and/or medical supplies in at least one transfer container (12), and for controlling the dispensing and/or use of drugs and/or medical supplies in at least one transfer container (12); and wherein the ID code of the remotely readable tag of the transfer container is read by the reader of the dispensing trolley (20) for recording the location of at least the transfer container (12) and/or the drugs and/or medical supplies contained in the transfer container.

4. The method according to claim 3, wherein personal data as well as data on the medication and treatment of a patient to be treated are entered in the data processing device (24) of the dispensing trolley (20), or in a data processing device with which a communication device in the dispensing trolley communicates; and these data are combined with the data on the drugs and/or medical supplies in the transfer containers (12) in the dispensing trolley (20), according to the patient's treatment regimen.

5. The method according to claim 4, wherein the dispensing trolley (20) is moved to the vicinity of the patient to be treated, and the reader of the dispensing trolley (20) is applied to read a remotely readable tag containing data of the patient and being worn by the patient or placed in the patient's bed or elsewhere in the vicinity of the patient, for identifying the patient and for administering the drugs intended for the patient to the patient, and/or for subjecting the patient to a medical treatment by using the medical supplies in the dispensing trolley (20).

6. The method according to claim 5, wherein the data processing device (24) in the dispensing trolley (20), and the computer program installed in it, or a communicating device arranged in the dispensing trolley and communicating with a data processing device elsewhere and a computer program therein, controls the dispensing of drugs and/or medical supplies placed in the transfer containers (12) after the patient's data have been read from a remotely readable tag worn by the patient.

7. A system for monitoring and controlling the use of drugs and/or medical supplies in a pharmaceutical dispensing process, the system comprising at least one storage device (10, 20) for drugs and/or medical supplies, and a data processing device (13, 24) connected to it or communicating with it via a communication device; a reader for remotely readable tags, connected to the data processing device (13, 24) or the communication device; a computer program installed in the data processing device and configured to read and store data on and quantities of drugs and medical supplies, to read ID codes contained in remotely readable tags and obtained from the reader, to combine ID codes with data on drugs and medical supplies, and to control the dispensing of drugs to patients and/or the use of medical supplies according to the treatment regimen; and the system comprising at least one transfer container (12) equipped with a remotely readable tag (16), the transfer container (12) being a container separate from the storage device (10, 20) and the product package of drugs and/or medical supplies, and removable from the storage device (10, 20).

8. The system according to claim 7, wherein the transfer container (12) is a container which can be fitted in a storage location (11) in the storage device (10, 20).

9. The system according to claim 7 or 8, wherein the transfer container (12) is a box or a case to be placed in a storage location (11) in the storage device (10, 20), having an inner space enclosed by at least one wall (12a to 12e), and a wall, or a part of a wall, that is open or equipped with an openable and closable lid via which the drugs and/or medical supplies can be transferred to the inner space formed by the walls (12a to 12e) of the transfer container.

10. The system according to claim 9, wherein the remotely readable tag (16) of the transfer container (12) is provided on at least one wall (12d) or openable lid of the transfer container.

11. The system according to any of the claims 7 to 10, wherein the remotely readable tag is an NFC or RFID tag.

12. The system according to any of the claims 7 to 11, wherein at least one storage device (10, 20) is a dispensing trolley (20) having at least one storage location (21) in which at least one transfer container (12) can be fitted and from which at least one transfer container (12) can be removed.

13. The system according to claim 12, wherein the dispensing trolley (20) comprises a data processing device (24) with a memory for storing and applying a computer program controlling the dispensing of drugs and/or the use of medical supplies, as well as for drug dispensing data, patient data, data on storage locations of transfer containers, and data on drugs and/or medical supplies contained in transfer containers.

14. The system according to claim 13, wherein the dispensing trolley (20) comprises a reader for remotely readable tags, connected to the data processing device, for reading the remotely readable tags (16) of transfer containers (12) to be transferred to the dispensing trolley.

15. The system according to any of the claims 12 to 14, wherein at least one storage location (21) in the dispensing trolley (20) comprises at least one indicating device connected to the data processing device (24) of the dispensing trolley (20), for indicating the storage location upon removing a transfer container from the dispensing trolley.

16. The system according to any of the claims 12 to 15, wherein at least one storage device (10, 20) is a handling tray having at least one transfer container seat for at least one transfer container (12); the handling tray comprising a data processing device, a computer program installed in it, and a reader for reading the remotely readable tag of the transfer container (12).

17. The system according to claim 16, wherein the handling tray comprises one or more indicating means for indicating dispensing units or the like contained in the transfer container (12) placed in at least one transfer container seat, for controlling the adding of dispensing units in the transfer containers (12) and/or the removal of dispensing units from the transfer containers (12).
